(19) United States
(12) Patent Application Publication
(10) Pub. No.: US 2004/0122475 A1
(43) Pub. Date: Jun. 24, 2004

(54) Electrochemical neuron systems

(76) Inventors: Andrew J. Myrick, Chicago, IL (US); James J. Myrick, Glencoe, IL (US)

Correspondence Address: James J. Myrick, 748 Greenwood Avenue, Glencoe, IL, 60022, US

(21) Appl. No.: 10/323,213

(22) Filed: Dec. 18, 2002

(51) Int. Cl. A61N 1/00

(52) U.S. Cl. 607/2

(57) ABSTRACT

The present invention relates to methods and systems for functionally stimulating or coupling neurons and neuron target cells to electronic circuits, which use a stimulating electrode and an ion selective zone adjacent the cell membrane of the cell to be stimulated which selectively absorbs and expels one or more stimulating ion species under control of the stimulating electrode. This changes the concentration of signal or response-controlling structures in the cell wall.

### REFERENCE TO PRIORITY APPLICATIONS

This application claims priority based on Provisional Application No. 60/216,224 filed Jul. 5, 2000 and PCT/US01/21233 filed Jul. 3, 2001.

### FIELD OF THE INVENTION

The present invention relates to methods and systems for functionally stimulating or coupling neurons and neuron target cells to electronic circuits.

### BACKGROUND OF THE INVENTION

Research and therapeutic technology involving the nervous system typically requires an effective signal interface with neurons or neuron target cells. A wide variety of devices such as heart pacemakers, cochlear implants, functional electrical stimulation (FES) systems, neural arrays and probes for the cortex, and retinal ganglia stimulator arrays are in use or under development. Cochlear implants use an array of electrodes inserted into the tympanic canal of the cochlea in close proximity to the neural pathways that innervate sound sensing hair cells in the organ of corti [G. Wang et al., “Unwrapping Cochlear Implants by Spiral CT”, IEEE Transactions on Biomedical Engineering 9 891-9 (1996)].

Another class of therapeutic systems which could be of enormous benefit to persons with spinal cord injuries, would replace damaged nerve systems through functional electrical stimulation (FES) [V. D. Chase, “Mind over Muscles”, March/April, http://www.techreview.com/articles/ma00/chase.htm (2000)]. FES would allow bypassing the spinal cord through the use of implanted electrodes to stimulate muscles in disabled limbs, or other target organs. For example, a joystick manipulated with the shoulder has been used to functionally stimulate muscles to control hand movement, and electrodes temporarily implanted in a primate's motor cortex can control robotic arms. If two-way communication could be re-established between severed spinal nerves, and the signals of multiple neurons “unscrambled” electronically for restimulation, major advances in rehabilitative medicine could be accomplished.

Electronic repair of vision is another important area of potential benefit. For example, certain degenerative diseases such as retinitis pigmentosa of the eye cause the photoreceptors in the retina to die, leaving the neural layer intact. Currently, experimental low resolution electrode arrays temporarily placed in contact with the retina can potentially be used to “project” images such as letters onto the retina of such patients. Limitations of such implants include long-term compatibility, and inefficient interaction, and the relatively high voltage or current used to achieve stimulation. Direct implantation of electrode arrays into the visual cortex of the brain has also been demonstrated to a limited degree, but much better electrode and interactive systems are necessary. Very low-power systems will be important, but available electrode systems have relatively high voltage and power requirements for nerve stimulation.

There has been a very significant amount of effort to develop and improve electrodes and electrode geometry, for example, through the use of etched wells in silicon to contain neurons. Microelectrode arrays (MEA) can be used to control action potentials in cultured neural networks and have been in use for many years as a tool for study. MEAs typically consist of a two dimensional array of exposed microelectrodes which are connected to conductors beneath an insulating layer. These conductors, which typically are of gold and/or platinum or other unreactive material to minimize electrochemical activity, can be connected to amplifiers and/or voltage/current sources in accordance with conventional practice. [G. R. M. Connol, et al., “Microelectronic and nanoelectronic interfacing techniques for biological systems”, Sensors and Actuators B 6 113-21 (1992); J. L. Novak et al., “Recording from the Aplysia Abdominal Ganglion with a Planar Microelectrode Array”, IEEE Transactions on Biomedical Engineering, BME-33 No. 2 196-202 (1986); U. Egert et al., “A novel organotypic long-term culture of the rat hippocampus on substrate-integrated multielectrode arrays”, Brain Research Protocols, 2 pp. 229-42 (1998)]. Some of the difficulties associated with such presently available MEAs involve their low signal to noise ratio and spatial-sampling related problems. Arrays have also been constructed for the purpose of monitoring peripheral nerves, in attempts to develop an effective, implantable peripheral nerve interface. Regeneration electrodes combine techniques [Chapter 42, Principles of Tissue Engineering (1997), R. G. Landes Co] used to guide damaged nerve endings with a microelectrode array. Silastic tubing bridging the gap between two ends of a transected or otherwise damaged nerve promote its regeneration up to 1 cm in length. The ends of the transected nerve will also regenerate through the holes in a perforated electrode array. [D. J. Edell, “A Peripheral Nerve Information Transducer for Amputees: Long-Term Multichannel Recordings from Rabbit Peripheral Nerves”, IEEE Transactions on Biomedical Engineering, BME-33 No. 2 203-14 (1986); G. T. A. Kovacs et al., “Silicon-Substrate Microelectrode Arrays for Parallel Recording of Neural Activity in Peripheral and Cranial Nerves”, IEEE Transactions on Biomedical Engineering, 41 NO. 6 567-77 (1994)] In addition to the problems associated with planar MEAs mentioned, such perforated MEAs can interfere with the normal regeneration process, cause constriction of axons in the regenerated fibers, or induce damaging mechanical stresses on the regenerated nerve fiber

There have also been efforts to develop more intricate neural interfaces for both stimulation and measurement of neural action potentials without application of net electrical current from electrodes. [Stett, A., et al. (1997) Two-way silicon-neuron interface by electrical induction, Physical Review Letters, 55 NO.2 1779-81; Fromherz, P., et al., (1995) Silicon-Neuron Junction: Capacitive Stimulation of an Individual Neuron on a Silicon Chip, Physical Review Letters, 75 1670-3; Weis, R., et al., (1996) Neuron Adhesion on a Silicon Chip Probed by an Array of Field Effect Transistors, Physical Review Letters, 76 327-30]. These devices function through a thin (e.g., 10 nm) capacitive gap between circuit elements and the intracellular fluid, to ameliorate adverse effects of electrochemical corrosion or reaction at the electrodes. Capacitive stimulation through this method relies on extremely intimate contact between the capacitive stimulation spot and the target cell. In addition, stimulation thresholds are unpredictable.

Unfortunately, cell-stimulating electrodes typically use relatively high levels of power (voltage and/or current). Amperometric electrodes can cause oxidation/reduction reactions as a result of the electrical current at the electrodes, which can produce toxic compounds and ions. Even capacitively-coupled electrodes may require a relatively high potential (e.g., a positive potential pulse or voltage swing of about 5 V) in order to induce an action potential in a target neuron. Not only can these applications of current and direct voltage applications be destructive over time, but the power requirement may be incompatible with the most effective therapeutic goals. For example, for direct current in vivo retinal cell stimulation, a minimum of 30-100 microamperes is used to produce action potentials in retinal ganglion cells, with electrode currents ranging up to a full milliampere being used for full scale stimulation at each electrode. Such power levels are potentially destructive and difficult to provide without external power sources. For example, water is hydrolyzed at electrode potentials over about 2 volts, and at such high voltages, chlorine ions can be oxidized at the electrode surface to produce toxic components.

Accordingly, there is a need for new methods and systems for stimulating cells that convey information through isotonic conduction such as bipolar cells, neurons, and neuron target cells such as muscle fibers and glandular cells. It is an object of the present invention to provide new technologies for stimulating and interacting with neurons and neuron target cells for research and/or therapeutic purposes. These and other objects of the invention (which need not all be accomplished in any one embodiment) will be apparent from the following detailed description and the accompanying drawings.

### SUMMARY OF THE INVENTION

The present invention is directed to ion-selective electrode systems devices and methods which can control concentration of selected ionic species such as potassium, sodium, calcium, chloride and/or hydrogen ions (pH) at a neuron or neuron target cell-electrode interface in order to affect cell membrane potential.

Neurons are highly specialized cells which generate and/or transmit impulses to a target cell such as another neuron, a muscle cell or an endocrine cell. The neuron contains within its cell membrane, a cell nucleus and a variety of organelles and other cytoplasmic inclusions. Extensions of the neuron cell membrane can connect with other neurons or neuron target cells for transmission of impulses through a synapse. Long tubular extensions of the cell membrane can form an axon to transmit impulses to neurons or other cells over a substantial distance. Depending on their function, human axons can typically range in length from around 0.1 millimeter to almost a meter in length (e.g., the sciatic nerve). Depending on the type of neuron and its function, its axons may branch into smaller extensions at its terminal end to form synapses with target cells (other neurons, muscle cells, glandular cells, etc.). Some axons, such as those in the peripheral nervous system, are partially insulated with a myelin lipid layer to speed and insulate neural impulses. This myelin sheath makes stimulation and sensing of such neurons more difficult.

The following information relates to the neuromuscular junction, describing a general mechanism for many types of synaptic transmission. Schematically, illustrated in FIG. 1 is a cross-sectional view of an axon terminal 102 of a presynaptic neuron, and a post-synaptic dendritic process 104 of a second, downstream, or “target” neuron. The presynaptic axon terminal 102 and the post-synaptic target neuron 104 are connected at a synaptic cleft 106. Ca⁺⁺, like Na⁺, is present in much higher concentration in the extracellular plasma fluid than inside the cell. When an impulse reaches the axon terminal 102, the depolarization of the terminal opens voltage-sensitive Ca ion (Ca⁺⁺) channels 108 in the cell membrane allowing calcium to diffuse into the cell and to trigger the release of a neurotransmitter such as acetylcholine from synaptic vesicles 110. In this regard, the calcium ions promote exocytosis of the neurotransmitter ACh into the cleft 106, where it diffuses to the postsynaptic membrane 112 of the postsynaptic neuron 104. The postsynaptic membrane contains a nicotinic ACh receptor for ACh. The nicotinic ACh receptor is a transmembrane ligand gated ion channel, that is opened when ACh binds to the extracellular portion of the ion channel protein complex 114. The neurotransmitter activates receptor sites 114 on the cell membrane 112 of the target neuron 104. The endplate potential is a very large depolarization (about 70 mV; compared with ≈1 mV for synaptic potentials at central synapses) and consistently raises the Vm of the muscle fiber from its resting value of about 90 mV, to the threshold for the firing of a muscle action potential. the firing threshold of the target neuron 104 is reached for depolarization of the neuron to produce a traveling action potential.

In order for the postsynaptic target neuron 104 to be able to depolarize and “fire”, it needs to be maintained at a characteristic internal negative electrical resting potential which polarizes its cell membrane 105. This resting potential, which is different for different types of cells, is achieved in large part by maintaining a relatively high concentration of sodium ions outside of the cell membrane, and a relatively high concentration of potassium ions in the cell, through the action of a selective sodium potassium pump system in the cell membrane. The sodium-potassium (Na⁺/K⁺) pump system, shown schematically at numeral 116 of FIG. 1, produces an approximately −70 millivolt difference between the negatively charged interior 107 of the illustrated neuron cell 104, and positively charged exterior. As shown in FIG. 1, the normal cell metabolism produces distribution gradients across the cell membrane for K⁺, Na⁺, Cl⁻, and Ca⁺⁺ (as well as other materials). As shown in FIG. 1, the relative permeability of a typical neuron varies with different inorganic anions which can affect its internal electrical potential (e.g., the relative permeability of K⁺≈1, Na⁺≈0.04 to 0.05, Cl⁻≈0.5). While there are some chloride ions shown within the cell, the internal concentration is less than the external chloride concentration, and anions are, to a large extent, phosphate and other and ionic components of the cytosol other than chloride. In addition, because the cell cytosol 107 and the external plasma solutions are ionically conductive, while the cell membrane is relatively non-conductive, the cell resting potential exists across the very thin cell membrane, producing a relatively high electrical field and therefore relatively high capacitance (e.g. about 1 uF/cm\hat{ }2).

The Na⁺/K⁺-pump 116 is a complex of proteins in the cell membrane that hydrolyses adenosine triphosphate (ATP), and uses the energy of the hydrolysis to pump potassium ions into the cell against the concentration gradient and to pump sodium ions out of the cell against the concentration gradient. For each two potassium ions pumped in, the protein system 116 pumps three sodium ions out of the cell. Neurons at rest may typically have intracellular sodium concentrations that are much less (e.g., about one tenth) than the extracellular sodium concentration, while their intracellular potassium concentration is greater (e.g., ten or more times higher) than the extracellular potassium concentration. The difference in permeability of the neuron membrane to sodium,potassium and chloride ions, and their concentration gradients and counter ion concentrations substantially produce the resting potential. As indicated in FIG. 1, the resting potential Vₘ of a neuron may be approximated by the standard Nernst-Goldman-Hodgkin-Katz (GHK) equation:

$${V}_{m} \approx - 61 {mVLog}_{10} [ \frac{( {P}_{k} {K}_{in} + {P}_{Na} {Na}_{in} + {P}_{Cl} {Cl}_{out} )}{( {P}_{k} {K}_{out} + {P}_{Na} {Na}_{out} + {P}_{Cl} {Cl}_{out} )} ]$$

where:

Pₖ is the cell membrane permeability (cm/sec) of the potassium ion,

P_{Na} is the cell permeability (cm/sec) of the sodium ion,

P_{Cl} is the cell permeability (cm/sec) of the chloride ion,

Kᵢₙ is the millimolar concentration of potassium ions in the interior (cytosol) of the cell,

Naᵢₙ is the millimolar concentration of sodium ions in the interior (cytosol) of the cell;

Clᵢₙ is the millimolar concentration of potassium ions in the interior (cytosol) of the cell;

Kₒᵤₜ is the millimolar extracellular concentration of potassium ions adjacent the cell membrane;

Naₒᵤₜ is the millimolar extracellular concentration of sodium ions adjacent the cell membrane; and

Clₒᵤₜ is the millimolar extracellular concentration of potassium ions adjacent the cell membrane.

Applying the GHK equation for typical neuron cell permeability ratios of Pₖ:P_{Na}:P_{Cl} of approximately 20:1:10, respectively, using typical neuron internal and external ion concentrations as shown below, results in a typical value of the neuron cell membrane potential Vm of −74 mV:

| | Potassium Ion | Sodium Ion | Chloride Ion |
|---|---|---|---|
| Internal cytosol | Kᵢₙ≈155 | Naᵢₙ≈12 | Clᵢₙ≈4.2 |
| concentration (mM) | | | |
| Extracellular fluid | Kₒᵤₜ≈4 | Naₒᵤₜ≈145 | Clₒᵤₜ≈123 |
| concentration (mM) | | | |

In an excitatory synapse, such as the neuromuscular junction, as neurotransmitter molecules attach to the receptor sites 114, ACh-sensitive ion channels 114 open at the cell membrane allowing sodium and potassium ions to pass through the membrane, to increase its internal voltage. Such an increase in potential is termed an excitatory post-synaptic potential (EPSP).A large enough EPSP caused by positive sodium ions entering the cell interior depolarizes the membrane, and triggers an action potential 124 that moves like a wave along the axon, as shown in FIG. 2. The propagation of an action potential is controlled by voltage sensitive sodium and potassium channels. Voltage-sensitive sodium channels 120 along the axon membrane respond to the EPSP by progressively opening, allowing sodium ions to enter the cell much more rapidly than when at rest. The influx of positively charged sodium ions causes the membrane to become even more depolarized. This added depolarization opens even more voltage-sensitive sodium channels 120, further depolarizing the membrane. This self-reinforcing process can cause the membrane potential to locally rapidly rise from, for example, about −70 mV, toward the opposite Nernst potential for sodium (+55 mV).

However, the Na⁺-driven membrane depolarization occurs for only a limited time because the voltage sensitive sodium channels 120 slowly become inactivated (close) of their own accord (see 120a) and pass no more Na⁺ current. It is estimated that in a typical cell, approximately 7000 sodium ions pass through each channel 120 during the brief period (about 1 millisecond) that it remains open. In addition, after a slight delay, the depolarization also opens voltage-sensitive potassium channels 122 that rapidly conduct potassium ions through the membrane, out of the cell. The voltage sensitive K⁺ channels (being of the delayed rectifier type), open more slowly than the voltage-sensitive sodium channels 120 after the membrane is first depolarized, so that the flow of K⁺ out of the cell occurs only after the Na⁺ inflow depolarization wave has raised the cell membrane potential. After the inward Na⁺ current wave has passed a depolarized zone along the axon membrane, the delayed inactivation of the voltage-sensitive sodium channels and the outward K⁺ current repolarize the membrane (the delay may be at least partially mediated by Ca⁺⁺ sensitive mechanisms). The electrical potential of the membrane action potential wave 126 traveling along the axon 104 away from the cleft 106, is plotted at the bottom of FIG. 2, in schematic registry with the axon 104.

When the action potential wave 126 reaches the end of the above example of a pre-synaptic bouton, the cycle is repeated; calcium channels are opened, and a neurotransmitter is released to a succeeding target cell. In addition to the Na⁺, K⁺, Ca⁺⁺ and acetylcholine-sensitive channels described, there are a wide variety of other ionic and ligand channels which are utilized in neuron sensing and transmission of information. There are a wide variety of neurons and neuron target cells with a very wide variety of ion-sensitive mechanisms which modulate their functioning. For example, while the binding of the neurotransmitter acetylcholine at certain synapse gates opens channels that allow Na⁺ and K⁺ to flow through the membrane to initiate a nerve impulse or muscle contraction, similar binding of gamma amino butyric acid (GABA) at certain synapses (e.g., GABA_{A} synapses in the central nervous system) opens channels to admit Cl⁻ or K⁺ ions into the cell (with ATP energy), which tends to inhibit the initiation of a nerve impulse. In motor neurons, such as illustrated in FIGS. 1-3, when one excitatory synapse 106 is activated, the depolarizing ionic currents initiate the traveling action potential wave or pulse. However, for other types of neurons, such as CNS neurons, small depolarizations or inhibitory systems at synapses all over the neuron membrane are dynamically summed together at a trigger zone to determine whether the neuron will fire an action potential.

Immediately after completion of the action potential wave, there is a brief refractory period when a second action potential cannot be triggered, while the voltage-sensitive sodium channels remain inactivated. An after potential 134 (FIG. 2), a hyperpolarization which follows the action potential wave and the absolute refractory period, is caused by the relatively slower closing of some of the voltage sensitive potassium channels. This after potential produces a relative refractory period of several milliseconds, during which a stronger depolarizing event than normal is required to trigger an action potential.

Isotonic conduction within the cell in part determines how fast an action potential can move along an axon with the rate of passive spread of potentials being approximately inversely proportional to both axoplasmic resistance and membrane capacitance. Fast action potential waves are enhanced in larger diameter axons which have reduced axoplasmic resistance, and by myelination of the axons of some types of neurons to reduce the membrane capacitance of the axon. The capacitance of the membrane depends upon membrane thickness because the electrostatic interactions responsible decline linearly with the distance separating the charge sheets (ions) collected on either side of the membrane. The thickness of the axonal membrane is greatly increased by myelination. During development, nerve accessory cells (Schwann cells) produce myelin sheaths approximately 1-10 microns in thickness (e.g., 3 microns) around the axon in lengths of, for example, about 200 microns. Between each myelinated segment are relatively short submicron-length segments of bare axon membrane, the Nodes of Ranvier. The action potential, generated by the opening of voltage sensitive sodium channels concentrated at the Nodes of Ranvier, is rapidly propagated through the myeliniated internode to the next Nodes of Ranvier segment, where the action potential wave is re-amplified by voltage-dependent opening of the high concentration of sodium channels at the node. In this “salutatory” conduction, the Node segments are primarily responsible for active generation of the action potential, which also reduces the energy required to maintain the concentration gradients of the sodium and potassium ions.

Unlike motor neurons or muscle fibers, individual central nervous system neurons typically receive synaptic inputs from many presynaptic neurons at multiple sites, and can in turn stimulate many target neurons. Each synaptic input may produce only a small synaptic potential (<1 mV). In addition, many of the synapses on each neuron are inhibitory rather than excitatory. Many different types of transmitter receptors are present which include both directly gated receptors (like the nicotinic AChR) and second messenger-coupled receptors. Glutamate is an excitatory transmitter for central nervous system (CNS) neurons, because most glutamate receptors cause inward (depolarizing) currents. There are a variety of different types of glutamate receptors that are ligand-gated ion channels; defined according to the drugs that specifically block them. These include AMPA types, NMDA types, as well as a secondary messenger-linked glutamate receptor (Quisqualate-B type). The AMPA type glutamate receptor channels produce the rapid early phase of the excitatory postsynaptic potential, while NMDA type receptors are responsible for a late contribution to the excitatory postsynaptic potential; both require glutamate binding and depolarization of the membrane before they open.

A primary inhibitory transmitter in some CNS neurons is gamma-aminobutyric acid (GABA), while an inhibitory transmitter for somatic motor neurons is glycine. These work mainly by opening Cl⁻ channels or K⁺ channels. Nernst potentials for Cl⁻ (and K⁺) are negative, so that opening of a ligand gated Cl⁻ channel can be used to inhibit depolarization by hyperpolarizing a neuron to make its resting potential slightly more negative, or can dissipate local stimulation currents before they reach the trigger zone of an axon hillock where CNS neuron action potentials are initiated. In this regard, some types of neurons may have a large number (e.g., 10,000) of presynaptic inputs, the summation of which determines whether the neuron will fire or not fire an action potential. Such highly interconnected neurons (not shown) typically have an axon hillock with both a high concentration of Na⁺ channels, and a lower threshold for sodium channel opening, which produces a “trigger zone” which is to the most sensitive portion of the neuron to the initiation of action potentials.

Accordingly, it will be appreciated that the stimulation and response of neurons (of which there are thousands of different types) and neuron target cells (including muscles, endocrine glands, heart, lungs, blood vessels, liver, fat deposits, exocrine glands, the gastrointestinal tract, adrenal medulla, kidney, urethra, bladder, sex organs, skin and eyes) is quite intricate and sophisticated, involving use of relatively small electrochemical potential gradients and concentration differences to sense, process and transmit information in an essentially nonelectronic manner. Conventional electrode systems which stimulate such cells by applying relatively high (and potentially destructive) electrical currents or electrical potentials, utilize a relatively crude, brute force approach which lacks compatibility with the biological functioning of the cells. In addition to adverse effects of high voltages and currents on the cells, it is also recognized that voltages in excess of about 2 volts can produce toxic materials such as atomic oxygen, hydrogen and chlorine. The methods and devices of the present invention provide electrochemical stimulation of neurons and neuron target cells and are more compatible with the biochemical function of the neurons and neuron target cells being stimulated. The term “stimulation” includes both excitatory and inhibitory stimulation.

As indicated, methods are provided for stimulating neurons or neuron target cells, which have protein gate structures in their cell membranes which are selective to one or more ionic species. Various aspects of such methods comprise the steps of providing a stimulating electrode and an ionically selective zone adjacent the cell membrane of the cell to be stimulated which selectively absorbs one or more of the selected ionic species. The desired ionic species are selectively absorbed in the ionically selective zone to create a reservoir of such ionic species. Preferably, the ion-selective zone will be at least twice as permeable to a particular selected ion, as it is to other aqueous ions with which it may be in contact. The ion selective zone(s) are placed adjacent the neuron or neuron target cell to be stimulated. An electrical potential is applied to the ion-selective zone to change the concentration of the selected ionic species in the ion-selective zone, and thereby the zone adjacent the cell membrane, to induce a biological response in the cell. The selected ionic species for the ion-selective zone(s) may be, for example, potassium, sodium, calcium, chlorine, and/or hydrogen ion (pH), and the stimulating electrode(s) may be a metal, electrically conducting organopolymer, or semiconductor electrode. Particularly preferred ionic species are potassium ions, and mixtures of potassium ions with a relatively small amount of calcium ions. The ion-selective zone may preferably be a layer of an organic or inorganic matrix containing an ionophore selective to the ionic species, although some porous glasses and other materials are themselves potassium-selective and may also be used. The ion selective zone may have an electrode immediately below it, or it may have an aqueous layer which can permit capacitive storage of ions against the electrode. The potential applied to the ionically selective zone may preferably be between + or − three volts with respect to the cell exterior, and may be considerably lower (e.g., preferably ±1 volt). The stimulating electrode may also be at least partially an electrically conducting organic polymer such as polypyrrole, polyaniline or polythiophene (including derivatives thereof), in which the stimulating electrode itself also forms the ionically selective zone. In this regard, for example, by applying a negative potential to a polypyrrole layer on an inert (e.g., gold) electrode in an aqueous electrolyte, the polypyrrole is reduced and cations may be incorporated from the electrolyte solution into the material. By applying a positive potential to the electrode, the polypyrrole is oxidized and the cations are expelled. Materials such as porous carbon and carbon aerogels may also be used as a cation reservoir layer, although such layers typically need a negative potential of, for example, −0.4 to −1.5 volts to incorporate cations for subsequent release [J. C. Farmer et. al., “Capacitive deionization of NaCl and NanO3 solutions with carbon aerogel electrodes”, J. Electrochem. Soc., Vol 143, No. 1, pp159-169 (1996)]. The negative, cation-reservoir-building potential may be applied with a slow rise time to minimize the effect on the adjacent neuron, and to facilitate capacitive reduction in the negative electric field “seen” by the neuron. Such carbon electrodes should also have an interconnected porosity in addition to the gel structure, to facilitate rapid ion movement in and out of the layer. Carbon aerogels may be readily formed in layers and fabricated into arrays using integrated circuit and MEMS micro-fabrication techniques.

Connexin proteins appropiate to and functionally compatible with a particular type of neuron may also be incroporated in the surface of the ion selective zone adjacent the neuron in use. The connexins may be incorporated and assembled directly in the ionophore polymer or in a lipid layer affixed to the ionophore polmer. Plant, A. L. Self assembled phospholipid/alkanethiol biomimetic bilayers on gold. (1993) Langmuir 9: 2764-2767; Plant, A. L., et al. (1994) Supported phospholipid/alkanethiol biomimetic membranes: Insulating properties. Biophys. J. 67: 1126-1133; Plant, A. L, et al. (1994) Planar phospholipid bilayer membranes formed form self-assembled alkanethiol monomers. Proceedings of the 13th Southern Biomedical Engineering Conference.; Plant, A. L., et al. (1995) Phospholipid/alkanethiol bilayers for cell surface receptor studies by surface plasmon resonance. Anal. Biochem. 226:342-348; Meuse, C. W., et al. (1998). Construction of hybrid bilayers in air. Biophys. J. 74. 1388.); Meuse, C. W., et al. (1998) Assessing the molecular structure of supported hybrid bilayer membranes with vibrational spectroscopies. Langmuir 14. 1604; Rao, N. M., et al. (1997) Characterization of biomimetic surfaces formed from cell membranes. Biophys. J. 73: 3066-3077; Matthias, M. F., et al., “Cell-free synthesis and assembly of connexins into functional gap junction membrane channels”, The EMBO Journal, Vol. 16, No. 10, pp. 2703-2716, 1997cells, ‘pair’ to form the complete, double-membrane Matthias M. Falk 1, Lukas; Rozenthal, R., et al., “Gap Junctions in the Nervous System”, Brain Res. Brain Res. Rev. 2000 April; 32(1): 11-5; Veenstra, R. D., “Size and Selectivity of Gap Junction Channels Formed from Different Connexins”, J. Bioenerg Biomembr, August 1996; 28(4):327-37; Ahmad, S., et al., “Cell-Free Synthesis and Assembly of Connexins into Functional Gap Junction Hemichaannels”, Biochem Soc Trans 1998 August: 26(3):S304; Falk, M. M., “Cell-Free Synthesis for Analyzing the Membrane Integration, Oligomerization, and Assembly Characteristics of Gap Junction Connexins”, Methods 2000 February; 20(2);'165-79

In the present invention, an ion-selective electrode zone or layer is used as a selective ion reservoir, in which discharge to, or incorporation of, the selected ion from a zone adjacent the neuron or neuron target cell is controlled by or modulated by potential (and/or current) applied to the ion-selective layer. Other aspects of the present invention are directed to ion-selective electrochemical stimulation systems. The ion-selective electrosynapses of the present invention use a solid membrane between a solid state electrode and the adjacent target cell, which is highly selective to particular ionic species, and which provide a voltage-controllable reservoir of the selected ions in the selective layer. In such electrochemical stimulation systems, the concentration of selected ions can be controlled by varying the potential applied to the reservoir electrode. By varying the voltage applied to the ion-selective reservoir layer, it is possible to modulate (selectively expel from, or incorporate into) the amount of the selected ion in the selective layer, and in a zone adjacent both the cell membrane and the ion-selective layer. In this way, the concentration of particular ionic species can be changed at the target cell surface. The desired selectivity of the selective layer may be provided by an ion selective agent such as an ionophore to increase the permeability of the selective layer to a specific ion. The ion selective layers may be formed from a plasticized amorphous polymer matrix, such as polyvinyl chloride or polyethylene-vinylacetate, which contains an ionophore selective for the ion desired to be controlled. For example, the ionophore valinomycin (a peptide antibiotic) may be incorporated into a polymer layer which is spun-cast on the microcircuit assembly to produce layers which are selective for potassium ions. Because of the relatively high cell membrane permeability of potassium ions as compared to sodium ions and chloride ions, relatively small changes in the potassium ion concentration at the exterior of the cell membrane cause relatively large changes in the cell membrane potential. Conversely, relatively large changes in the sodium ion and chloride ion concentration outside the cell membrane have a relatively small effect on the membrane potential, compared to the effect of a potassium ion concentration change.

For example, using the previously-discussed GHK approximation equation of FIG. 1, an increase of only 20 mM of K⁺ ion concentration at the membrane outer surface causes an increase, ΔVm, in the membrane potential of 24 mV, while a 20 mM increase in Na⁺ concentration produces a decrease of only 2 mV and decrease in the Cl⁻ concentration at the outside of the cell membrane only causes a decrease of 1 mV in the cell membrane potential. The membrane potentials for these external ion concentration changes in the previously described neuron initially having resting cell permeability ratios of Pₖ:P_{Na}:P_{Cl} of 20:1:10, respectively, and are shown in the following Table:

| | Potassium Ion | Sodium Ion | Chloride Ion |
|---|---|---|---|
| Internal cytosol | Kᵢₙ = 155 | Naᵢₙ = 12 | Clᵢₙ = 4.2 |
| concentration (mM) | | | |
| Extracellular fluid | Kₒᵤₜ = 4 | Naₒᵤₜ145 | Clₒᵤₜ = 123 |
| concentration (mM) | | | |

| Outside | Outside | Outside | | | Vm above the |
|---|---|---|---|---|---|
| K⁺ | Na⁺ | Cl⁻ | Vm | | −55 mV firing |
| (mM) | (mM) | (mM) | (mV) | ΔVm | potential? |
| 4 | 145 | 123 | −74 | 0 (resting | no |
| | | | | Vm) | |
| 24 | 145 | 123 | −50 | 24 mV | yes |
| (increase) | | | | | |
| 4 | 165 | 123 | −72 | 2 mV | no |
| | (increase) | | | | |
| 4 | 145 | 103 | −73 | 1 mV | no |
| | | (decrease) | | | |

Accordingly, inducing a relatively small local change in the concentration of potassium ions at the outside of an active neuron cell membrane by modulating the potassium ion content of an ion-selective reservoir zone adjacent the membrane, can increase the local membrane potential of neuron 104 from its resting value of about −74 mV, by at least the approximately +20 mV necessary to reach a neuron “firing” initiation potential which is more positive than about −55 mV. In fact, K+ solutions as dilute as 10 mM are conventionally used to depolarize neurons in laboratory test procedures [K. Morita et al, “Posttetanic hyperpolarization produced by electrogenic Na(+)-K+ pump in lizard axons impaled near their motor terminals”, J. Neurophysiol 70(5):1874-84 (1993)]. It is important to note that this strong depolarizing effect is different from the external local electric field of the electrode, which also may be significant and complementary to the depolarizing effect.

An important concern involving the action potential induction by this method is the time scale of the voltage change resulting from a change of extracellular potassium. The change in the membrane voltage can be characterized with a time constant that represents the time it takes for the membrane to reach 63% of its final change in voltage. Typical cell membrane capacitance is C=1 uF/cm\hat{ }2, while typical resting membrane conductivity is R=50 KΩ-cm\hat{ }2. The time constant of the membrane, RC=50 ms, is not affected (using a simple model of membrane conductivity) by the potassium concentration change. This compares to synaptic transmission times of approximately 1-3 ms.

While potassium is the particularly preferred ion for concentration modulation to initiate neuron or neuron target cell firing, the concentration of other ions may also be modulated to stimulate neurons and other cells. Appropriate ionophores may be used to produce layers which are selective to other ions such as sodium, calcium and chlorine ions, which as indicated above, are also important to (e.g., sensing and triggering) various neuron functions. For example, nonensin (and its covalently bondable derivatives) may be used to prepare layers which are selective to sodium ion transport, while tridodecylamine (and its covalently bondable derivatives) can similarly be incorporated in polymer layers to produce layers which are selective to transfer of hydrogen ions.

This use of ion-selective electrochemical electrode stimulation is illustrated schematically in FIG. 3, which shows the neuron segment 104 adjacent an integrated circuit electrode array 150. The electrode array 150 is fabricated using photolithographic integrated circuit manufacturing techniques on a suitable substrate 152 such as silicon or polyimide. A series of individually controllable electroconductive electrodes 154, 156, 158 (e.g., 0.5 to 30 microns in width) is fabricated and positioned atop the substrate 152. Covering electrode 154 is a potassium ion selective membrane 160. Covering electrode 156 is a potassium-selective membrane 162, and covering electrode 158 is a chloride-ion selective membrane 164. Before applying an electrochemical pulse to the neuron 104, electrode 154, the electrodes may be at zero potential (or may be biased 0.1-0.5 volts negative). Electrode 156 may initially similarly be held at zero potential, or a very slightly negative potential vs. the extracellular fluid 101. The potential of the Cl⁻ selective electrode-modulated selective reservoir layer may initially also be at zero potential or at a very slightly positive potential of less than 0.5 volts vs the extracellular fluid 101. The duration of the application of these potentials may be controlled as desired (with the electrical field lines diminishing quickly over time as the ionic components readjust their position and concentrations in response to the field). Typically, the duration may be from 0.05 to about 50 milliseconds. The “instantaneous” membrane potential initially induced by the application of a relatively small positive potential to electrodes 154 and 156, and a relatively small negative potential to electrode 158 is shown schematically by curve 170 in registration with the neuron 104 and the microcircuit 152.

The specific voltages applied will depend on the distance between the neuron 104 and the electrode array 152, as well as other factors such as the ionic and electrical properties of the extracellular fluid in the zone 101, and the neuron itself. Preferably, the potential applied to the control electrode to induce stimulation of a neuron action potential will be less than 2 volts, and more preferably less than 1 volt. It is important to note that this voltage may be substantially less than that required to induce neuron firing in the absence of the ionic stimulation produced by the modulation of the ion-reservoir layers in accordance with the present invention. In addition, the increase in potassium ion concentration induced by the modulation of ion-selective reservoir layers such as 160, 162 can be less than that required for inducing an action potential by this mechanism alone. To minimize the power and voltage requirements (and adverse effects on the cell 104 and surrounding cells), it is preferable to design the electrochemical synapse system so that from about 10 to about 90 percent (e.g., 25-75%) of the localized neuron depolarizing effect is caused by the electric field from the electrode such as 154, 156, and from about 90 to about 10 percent (e.g., 75-25%) of the depolarizing effect is caused by the change in ion concentration at the cell membrane as a result of concentration modulation of the ion-selective layer adjacent thereto. The design of the electrochemical synapse system includes selection of electrode and ion-selective layer size (primarily width and thickness, respectively), the distance between the cell membrane and the electrode and ion-selective layer surfaces, and the amount of ionophore in the ion-selective layer.

A gap (e.g., 3-5 microns) is shown between the neuron 104 and the ion selective layers in FIG. 3 to accommodate a myelin sheath (e.g., 3 microns), or other separation distance, which somewhat increases the required stimulation voltages. Desirably, the ion-selective reservoir layer should be positioned within 15-20 microns or less of the cell membrane. The ability of the ion-selective reservoir to change the concentration of the selected stimulatory ion at the cell membrane surface depends on both the amount of the selected ion which can be stored and modulated by the ion-selective layer, and the volume of the (aqueous) zone between the cell membrane surface and the ion-selective layer. Accordingly, the ion-selective reservoir layers will preferably be designed and fabricated to emphasize maximization of the amount of the selected ion stored per unit volume, rather than other factors such as the Nernst potential. For example, for typical use as an analytical electrode, the potassium-selective ionophore valinomycin may be incorporated at a level of 1-2 weight percent in the measurement electrode layer to maximize voltage sensitivity. However, to increase the concentration of potassium ions in the ion-selective reservoir layers 160, 162, larger amounts of the ionophore (e.g., 5-25% by weight based on total dry weight) may be incorporated. In addition, the ion-selective layer will preferably be at least about 2 microns in thickness, and will preferable be positioned within a distance to the cell membrane of less than or equal to its own thickness. Preferably, the neuron will be at least partially adherent to the ion-selective surface, which greatly reduces the potentials used. In addition, in order to reduce the time for the ions to be discharged from the ion-selective reservoir layer, it is preferred that the layer have at least about 5 volume percent interconnected porosity (e.g., having a pore diameter of 10-500 nanometers), communicating with the exterior surface, to facilitate rapid expulsion (and incorporation) of the selected ions.

As will be described, the ionic-reservoir zone stimulation may be applied in a manner which is synergistic with the depolarizing effect of the controlling electrode potential. When a positive voltage or current source is applied to electrodes 154, 156 the neuron is stimulated by driving potassium ions from the selective layers 160, 162 to locally increase the concentration of K⁺ ions at the nerve cell surface, and decreasing the potential of the cell membrane to stimulate the Na⁺ sensitive gates 120. The internal potential of the cell is locally decreased in the vicinity of the electrode by the positive electric field of the electrodes. After the injected potassium has diffused around the cell, the electric field may be reversed rapidly and for a short period of time, causing the cell to depolarize in the vicinity of the electrode. Less voltage is required to stimulate the neuron to firing than would be the case if a capacitive or amperometric electrode alone is used to stimulate the neuron. The negative voltage may be applied to the electrode(s) 154, 156 for at least about 0.1 to 2 milliseconds (e.g., 1-50 milliseconds) to accomplish the desired stimulation effect consistent with the cell time constants. If a positive potential is simultaneously applied to the electrode 158, the neuron is further stimulated by establishing a sharp voltage gradient along the axon which drives the lateral redistribution of ions along the cell (both at the inside and at the outside of the cell membrane). Relatively sharp lateral potential gradients 166 at the cell membrane can be produced by closely spaced adjacent electrodes 156, 158 (e.g., 1-5 micron spacing for 2-20 micron wide electrodes). The potentials of the electrodes 154, 156, 158 may be returned to zero volts, and succeeding electrodes (not shown) may then be similarly pulsed, and timed in the direction and rate of intended nerve pulse travel. The polarities of a multiplicity of electrodes may be peristaltically clocked along the nerve to stimulate it or inhibit it and influence its response along its length, as desired.

### DESCRIPTION OF THE DRAWINGS

FIG. 1 is a schematic cross-sectional view of a presynaptic neuron, a post-synaptic neuron, and the synapse between them illustrating the neuron resting potential and presynaptic stimulation;

FIG. 2 is a schematic cross-sectional view of the postsynaptic neuron of FIG. 1, illustrating the progression of an action potential along the postsynaptic axon;

FIG. 3 is a schematic cross-section of the post-synaptic neuron of FIGS. 1 and 2, under stimulation from an embodiment of an ion-selective electrochemical electrode array in accordance with the present invention;

FIG. 4 is a schematic cross-sectional view longitudinally along the axon of an embodiment of a multi-ion-selective neural interface, with K⁺, Ca⁺⁺ and/or Cl⁻ ion-selective electrode neuron stimulation zones;

FIG. 5 is a cross-sectional view, transversely across the axon, of and the electrode chemical neuron stimulation system like that of FIG. 4, including floating-gate ion selective electrochemical stimulator-sensor electrodes which are able to both stimulate and sense the activity of a neuron;

FIG. 6 is a cross-sectional view of a retinal photodiode electrode implant with ion-selective electrode coatings;

FIG. 7 is a block diagram of a multineuron interconnection array using intermediate digital logic and electronic signal processing;

FIG. 8 is a top view of a silicon muscle innervation array layer test system for muscle stimulation study

FIG. 9 is a side view of the three layers of the silicon-muscle innervation array of the test system of FIG. 8.

FIG. 10a is a cross-sectional side view of a MEMS-type peristaltic ion pump having a thin organopolymer aqueous ion conduction channel with CCD-type drive electrodes for controlling the electric field and transporting ions in the channel;

FIG. 10b is a cross-sectional side view of the peristaltic ion pump of FIG. 10b, illustrating progressive transport of ions along the thin organopolymer channel under multi-phase CCD-like transport control of the drive electrodes.

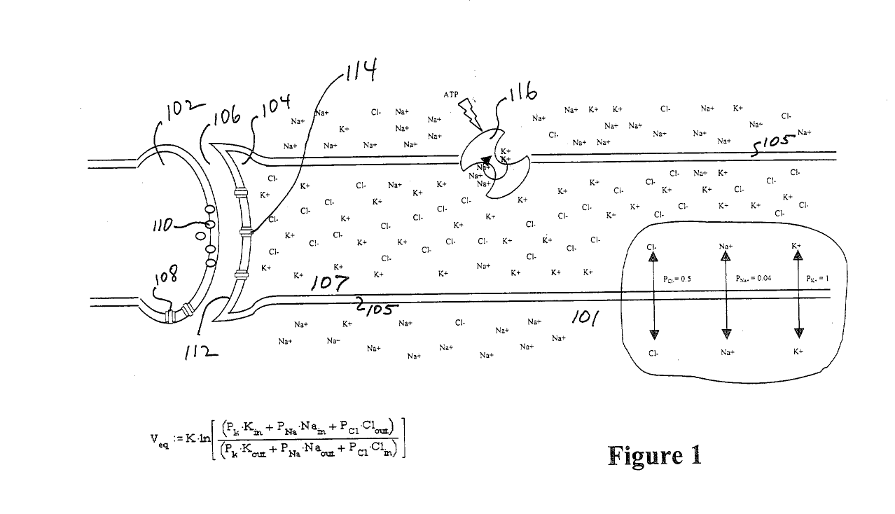

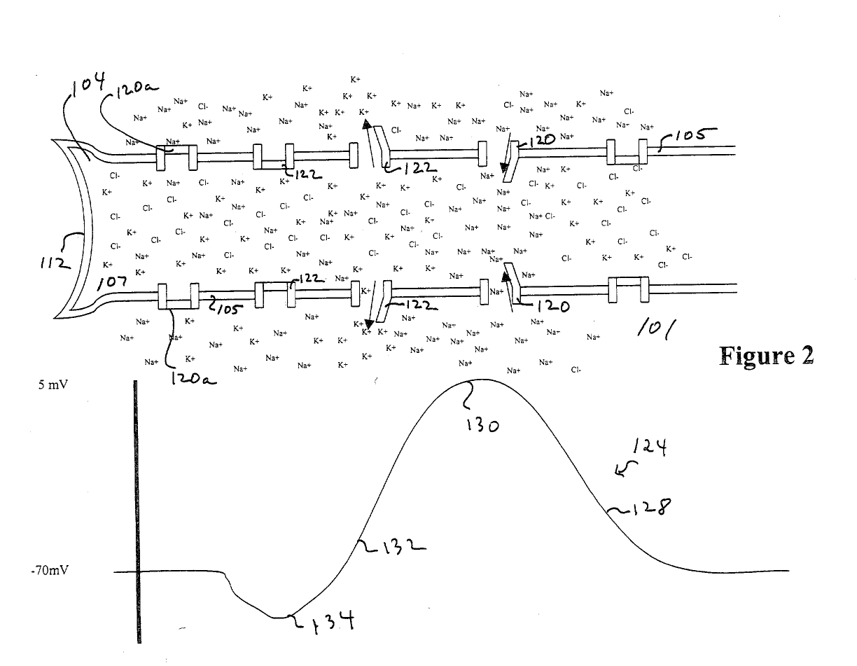

### DETAILED DESCRIPTION OF THE INVENTION

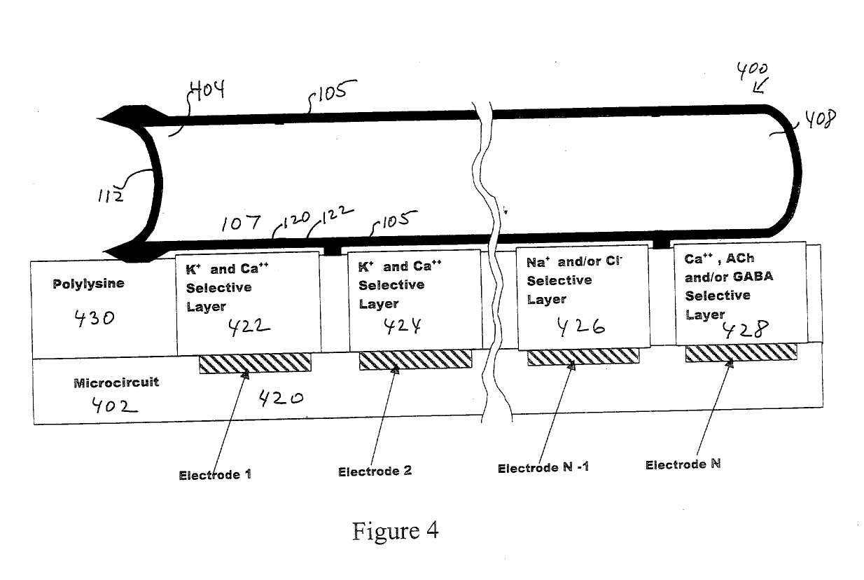

As previously discussed, the present invention is directed to ion-selective electrochemical neuron stimulation systems, including methods and devices which can control both electrical potential and concentration of selected ionic species such as potassium, sodium, calcium, chloride, nitrate, nitrite, carbonate and/or hydrogen ions (pH) at a neuron or neuron target cell-electrode interface. In this regard, illustrated in FIG. 4 is an embodiment of an ion-selective electrochemical neuron stimulation system 400 which comprises an integrated microcircuit 402 having ion-selective reservoir layers, and an “attached” neuron 404. The neuron 404 (which may be further functionally connected to other neurons work target cells, as well as other microcircuit components in a neural network system), typically has one or more presynaptic junctions 406, and an end terminal 408 for transmission of a stimulating signal to one or more subsequent neurons or target cells. The neuron 404 has, as is typical, a large number of voltage-dependent ionic gates, including potassium-selective gates in its cell membrane 105 adjacent the microcircuit 402. The microcircuit 402 comprises a plurality N of individually controllable electrodes 1,2, . . . , N-1, N which are fabricated on a suitable substrate 420 and electrically isolated from each other. Atop each electrode is an ion-selective reservoir layer, which may include as shown in FIG. 4, potassium-ion-selective layers 422, 424 atop electrodes 1, 2, a sodium ion and/or chloride ion selective layer 426 atop electrode N-1, and a calcium and/or ACh and/or GABA-selective layer 428 atop electrode N, adjacent a synapse. The ion selective layers 422, 424, 426, and 428 may be in direct electrical contact with the conductive metal or polysilicon electrodes 1,2, . . . , N-1, N adjacent thereto in an amperometric electrode system, or they may be separated from the electrodes by a very thin dielectric layer (not shown) such as a silicon dioxide or silicon nitride layer in a capacitive-coupled system. Many ion-selective layers are relatively poorly conductive, and accordingly also can support a capacitive function.

The ion-selective layers may be fabricated on the underlying electrode array by coating a suitable ion-selective layer on the microcircuit array, followed by photographic etching (preferably plasma-etching for both inorganic-based and organopolymeric-based ion selective layers). The layers 422, 424, 426 and 428 may vary widely in thickness, but typically may be in the range of from about one to about 10 microns in thickness. The electrodes and ion-selective layers may be provided in a wide range of widths (shown here along the neuron axis), for example, 0.1 to 50 microns, preferably 2 to 20 microns. A polylysine or other neuron-attachment-promoting layer 430 (similarly from 0.1 to 10 microns and thickness) may be deposited by solution or other appropriate methods. This layer may be used to guide the position of the neuron, but need not cover the ion-selective zone which is adjacent the neuron (it is desirable that there be some interchange with the plasma for equilibration purposes). Preferably, this neuron-adherence layer is covalently bonded to the microcircuit substrate, and is lightly crosslinked in order to promote longevity and stability of the layer. Other physiologically appropriate materials may be used as coating or adjacent layers, as desired [e.g., Brooks, et al., “Effect of surface-attached heparin on the response of potassium-selective electrodes”, Anal Chem Apr. 15, 1996: 68(8):1439-43]. Electrodes may typically be fabricated from metals and semiconductors, but electrodes and ion-selective membrane layers may also be fabricated from conjugated aromatic polymers such as polypyrroles, polyanilines and polythiophenes. [e.g., see R. E. Gyurcsanyi et al., “Novel polypyrrole based all-solid-state potassium-selective microelectrodes”, Vol. 12, Iss. 006, pp. 1339-1344 (1998); M. Yamamoto et al., “Evaluation Of Electrode Characteristics Of All Solid State Potassium-Selective Electrode Using Polypyrrole/Polyanion Composite Film”, Proceeding of the 19th Chemical Sensor Symposium, Vol. 10, Supplement B), Sep. 19-20, 1994 Keio University, pp125-128 (1994)]. Photolithographic and integrated circuit manufacturing techniques used to create electrical circuits in silicon, are well-suited for integration of neurons and electronic silicon structures in accordance with the present invention, and to pattern the adhesion of neurons to silicon based substrates. [D. Keinfeld et al., “Controlled Outgrowth of Dissociated Neurons on Patterned Substrates”, The Journal of Neuroscience, 8(11) pp. 4098-4120 (1996); M. Matsuzawaet et. al., “Containment and growth of neuroblastoma cells on chemically patterned substrates”, Journal of Neuroscience Methods, 50, pp. 253-260 (1993); J. M. Corey et al., “Micrometer resolution silane-based patterning of hippocampal neurons: critical variables in photoresist and laser ablation processes for substrate fabrication”, IEEE Transactions on Biomedical Engineering, 43(9):944-55 (1996);B. Palan, et al., “New ISFET sensor interface circuit for biomedical applications”, Sensors and Actuators B 57 (1999) pp. 63-68; M. Schwank, et al., “Production of a microelectrode for intracellular potential measurements based on a Pt/Ir needle insulatsed with amorphous hydrogenated carbon”, Sensors and Actuators B 56 (1999) pp. 6-14; S. Hediger, et al., “Fabrication of a novel microsystem for the electrical characterisation of cell arrays”, Sensors and Actuators B 56 (1999) pp. 175-180; R. Garjoynyte, et al., “Glucose biosensor based on glucose oxidase immobilized in electropolymerized polypyrrole and poly(o-phenylenediamine) films on a Prussian Blue-modified electrode”, Sensors and Actuators B 63 (2000) pp. 122-128; W. H. Baumann, et al., “Microelectronic Sensor system for microphysiological application on living cells”, Sensors and Actuators B 55 (1999) pp. 77-89]. Cell surface receptors may also bind to certain amino acid sequences in the extracellular matrix (ECM) protein, laminin, which may also be patterned for neuron growth control by microfabrication techniques. Binding of these receptors can achieve patterned neural attachment and outgrowth. [I. Yoshihiro, “Surface micropatterning to regulate cell functions”, Biomaterials, 20, pp. 2333-42 (1999); P. Clark et al., “Growth cone guidance and neuron morphology on micropatterned laminin surfaces”, Journal of Cell Science, 105, pp. 203-212]. The ion-selective layers 422, 424, 426 are predominantly potassium-selective layers, but may contain a small amount of a calcium ionophore (e.g., less than five weight percent of the potassium ionophore content, or an amount which produces a calcium content of less than five percent by weight of the potassium content within the selective layer) if desired for use with neurons in which calcium ions play a role in the opening of the sodium ion gates. In systems in which the neuron cell membrane adheres to the ion-selective layer, it may be desirable to permit the ion selective layer to admit a small amount a sodium ions as well; however, the ion selective layer should preferably maintain a molar concentration of potassium ions exceeding that of sodium ions.

Many cellular functions are activated by ions other than potassium, such as calcium and chloride. For example, calcium can enter cells through specialized proteins (largely voltage-independent-calcium channels) in the cell membrane, to modulate various cell functions. Such voltage independent calcium channels are found in high density in a number of different neurons and muscles. The present invention permits stimulation of even such voltage independent channels by local variation of the calcium concentration at the cell membrane, by using calcium selective layers under electrode control as shown in FIG. 4. There are a wide variety of neuron target cells (any cell which is a stimulation target) with a very wide variety of ion-sensitive mechanisms which modulate their functioning, which may be stimulated in accordance with the present disclosure. In this regard, the illustrated embodiment also has a calcium and/or chloride-ion-selective electrochemical electrode at the terminal end of the neuron 404, so that either calcium or chloride ions can be applied at the neuron terminal, with an outward (relative to the electrode) directed or inward directed electrical field, respectively. As indicated, the voltage modulated ion-selective reservoir layers are an important aspect of the present invention. Suitable layers may be made by incorporating a suitable ionophore into a matrix material, applying the ionophore and its matrix to the electrode array, followed by photolithographic patterning. It is also possible to first form the ion-selective zone(s) or layer(s), and to subsequently fabricate the electrode(s) on the ion selective material.

Suitable matrices may be either organic or inorganic. Organopolymers suitable for formation of ion selective membranes include polyvinylchloride, polystyrene, polyacrylate, polycarbonates, silicone resins, polyesters, polyamides, vinylidene chloride, acrylonitrile, polyurethanes, polyvinylidene chloride, polyvinylidene chloride copolymerized with polyvinylchloride, polyvinyl butyryl, polyvinyl formal, polyvinyl acetate, polyvinyl alcohol, polystyrenes, polyacrylonitriles, polyacrylamides, cellulose esters, potycarbonates, polyurethanes, epoxy resins, polyester resins, polyvinyl butyral resins, acrylic resins and methacrylic resins (including hydroxyethers and derivatives), and partially sulfonated poly—[perfluorinated ethylene]—(Dupont Nafion brand products)and copolymers of the such materials. In some embodiments, plasticizers may be used in the preparation of the ion selective layer, such as dimethylphthalate, dioctylphenyl-phosphonate, dibutylphthalate, hexamethylphosphoramide, dibutyladipate, dioctylphthalate, diundecylphthalate, dioctyladipate, dioctyl sebacate, and other conventional plasticizers. [see also—U.S. Pat. No. 4,434,249; C. L. Ballestrasse et. al., “Acrylic Ion-Transfer Polymers”, Journal of the Electrochemical Society, 134, 11, 2745-2749 (1987)]

Suitable inorganic matrices include inorganic silicate, titanate, zirconate and similar oxide-gel materials which may be formed by slow, controlled hydrolysis of the respective silicon, titanium, and/or zirconium, organoethers such as their tetraethyl or tetramethyl ethers/esters.

In fabricating the ion-selective layer, a suitable ionophore is included with a matrix-forming material, which may then be applied (e.g., by spin-coating) to the electrode array. Desirably, the ionophore component will comprise from about 1 to about 50 weight percent of the ion selective layers.

Ion selective materials may be any of those known to be selective towards the particular ion to be controlled. For potassium-selective storage layers, valinomycin, bis(benzo-15-crown-5)-4-ylmethyl)pimelate, dicyclohexano-18-crown-6, benzo-18-crown-6,dibenzo-30-crown-10, biscrown ethers and biscrown ethers such as 2,2-bis>3,4-(15 crown-5)-2-nitrophnylcarbamooxymethyl!tetradecanol-14, dibenzo-18-crown-6, tetraphenyl borate, tetrakis(p-chlorophenyl)borate, cyclopolyethers, tetralactones macrolide actins (monactin, nonactin, dinactin, trinactin), the enniatin group (enniatin A, B), cyclohexadepsipeptides, gramicidine, nigericin, dianemycin, nystatin, monensin, esters of monensin, and/or antamanide, alamethicin (cyclic polypeptides) may be used as ionomers. For calcium, ETH1001 (as disclosed in Anal. Chem. 53, 1970 (1981), ETH129, A23187 (Fluka of Buchs, Switzerland), ETH5234 (Fluka of Buchs, Switzerland), bis(didecylphosphate), bis(4-octylphenylphosphate), bis(4-(1,1,3,3-tetramethylbutyl))phenylphosphate, tetracosamethylcyclododecasiloxane, N,N′-di((11-ethoxycarbonyl)undecyl)-N,N′4,5-tetramethyl-3,6-dioxaoctane diamide, antibiotic A-23187 (as disclosed in Ann. Rev. Biochem. 45, 501 (1976)); for hydrogen, ETH1907 (Fluka of Buchs, Switzerland), ETH1778 (Fluka of Buchs, Switzerland), tridodecylamine, ETH1907, N-methyl n-octadecyl (1-methyl, 2-hydroxy, 2-phenyl)ethylamine, N-octadecyl3-hydroxyNpropylamine, N,N′bis(octadecyl ethylene amine), p-octadecyloxy-m-chlorophenylhydrazonemeso oxalonitrile may be used as an ionomer component in the ion-selective calcium storage layer. Monensin, ETH227 (Fluka of Buchs, Switzerland), ETH157 (Fluka of Buchs, Switzerland), ETH4120 (Fluka of Buchs, Switzerland), ETH2120 (Fluka of Buchs, Switzerland), ETH227, ETH157, NAS.sub.11-18, N,N′,N″-triheptyl-N,N′,N″-trimethyl-4,4′, 4″-propylidintris-(3-oxabutyramide), 4-octadecanoyloxymethyl-N,N,N′,N′-tetracyclohexyl-1,2-phenylenedioxydiacetamide, bis>(12-crown-4)methyl!dodecylmethylmalonate, ETH149, ETH1810, cyclopolyethers; for lithium, N,N′-diheptyl-N,N′,5,5-(tetramethyl-3,7-dioxononanediamide), ETH149 (Fluka of Buchs, Switzerland), ETH1644 (Fluka of Buchs, Switzerland), ETH1810 (Fluka of Buchs, Switzerland), ETH2137 (Fluka of Buchs, Switzerland), 6,6-dibenzyl-14-crown-4; 6,6-dibenzyl-1,4,8,11-tetraoxa-cyclotetradecane (Fluka of Buchs, Switzerland), 6>2-(diethlphosphonoox)ethyl!6-dodecyl-14-crown-4 (Fluka of Buchs, Switzerland), 12-chrown-4,6,6-dibenzyl-14chrown-4, cyclopolyethers, and dodecylmethyl 14-crown-4 may be used as a sodium-selective ionomer for sodium ion-storage layers. Tridodecyl ammonium salt, 5,10,15,20-tetraphenyl-21H, 23H-porphin manganese (111) chloride (Fluka of Buchs, Switzerland), quaternary ammonium chloride and tributyl tin chloride may be used for chloride-selective storage layers. Other ETH compounds are disclosed in Anal. Sci. 4, 547 (1988).

Other examples of ion-selective ionophores (including covalently bound species) and layers and solvents therefore are described in U.S. Pat. Nos. 4,214,968, 3,562,129, 3,753,887, and 3,856,649, 4,554,362, 4,523,994, 4,504,368, 4,115,209, 5,804,049, EP 0 267 724, WO 91/1171 0 (Aug. 8, 1991), Daunert et al., Anal. Chem. 1990, 62, 1428-1431, and Oue, M. et al., J. Chem. Soc.-Perkin Trans. 1989, 1675-1678.

Zeolitic materials also have ion-selective properties, and may be incorporated in a suitable inorganic or organic matrix for application to an electrode or electrode array, and/or may be formed in situ on the electrode(s). Ion exchange resins, such as cationic exchange resins which exclude anions (e.g., crosslinked polystyrenesulfonate layers) can provide crude cation selectivity over anions at the control electrode, and anionic exchange resins (e.g., crosslinked polyaminostyrene layers) can provide crude anion selectivity over cations in the extracellular fluid, but are not preferred to provide the precise, targeted stimulation of specific ion-selective layers.

Because of the relatively large changes in cell membrane potential which can be caused by relatively small changes in the extracellular potassium ion concentration, electrode systems with potassium-selective layers are particularly effective for neuron stimulation. Valinomycin cyclo-[-D-valine-L-lactic acid-L-valine-D-.alpha.-hydroxyisovaleric acid]) is a preferred ionophore that makes the layer selectively permeable to potassium. It is a natural dodecadepsipeptide of neutral ionophore twelve alternating amino acids and hydroxy acids (D- and L-valine, D-hydroxyvaleric acid and L-lactic acid) which form a macrocyclic molecule:

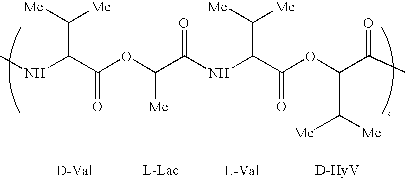

Valinomycin, like the various crown ethers (e.g., 18-crown-6 cyclic ether) is highly selective towards potassium ions over sodium ions. When enclosing a potassium ion, valinomycin forms a cage configuration with the isopropyl groups at the cage exterior. Accordingly, functional valinomycin derivatives for potassium-selection may be provided in which one of the external groups, such as one of the isopropyl groups, is substituted by a vinyl-polymerizable group, such as a pendant acryloyl or methacryloyl group with an intermediate ethylene oxide moiety to improve conformal flexibility in the copolymerized form. The pendant acryloyl or methacryloyl groups copolymerize with vinyl-polymerizable or cross-linkable polymers. Upon copolymerization with other vinyl-polymerizable co-monomers in the matrix, the ionophore may be formed as an integral, permanent (non-leachable) part of an ion-selective layer. Photopolymerizable systems are also desirable. For example, potassium-ion-selective layers based on an epoxyacrylate polymer prepared from o-nitrophenyl octyl ether as the plasticizer and 2,2′-bis[3,4-(15-crown-5-)-2-nitrophenylcarbamoxymethyl]tetradecane (BME-44) as the ionophore may be applied as a photocurable layer over an electrode. [P. W. Alexander, et al., “A Photo-Cured Coated Wire Potassium Ion-Selective Electrode for Use in Flow Injection Potentiometry”, Electroanalysis, 9 (11) 813-817 (1997)]. Other ionophores may be similarly covalently bonded in a matrix.

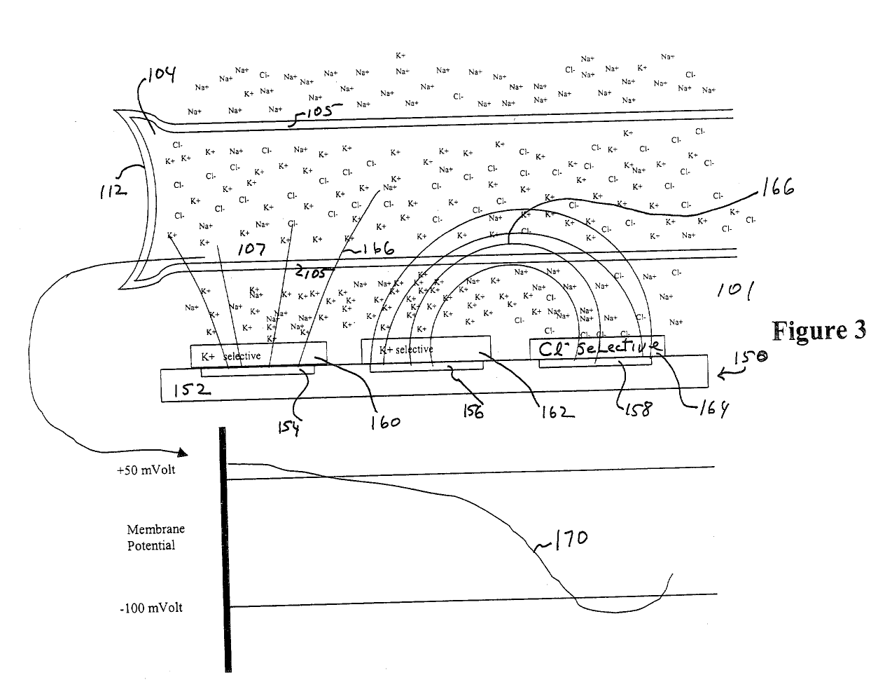

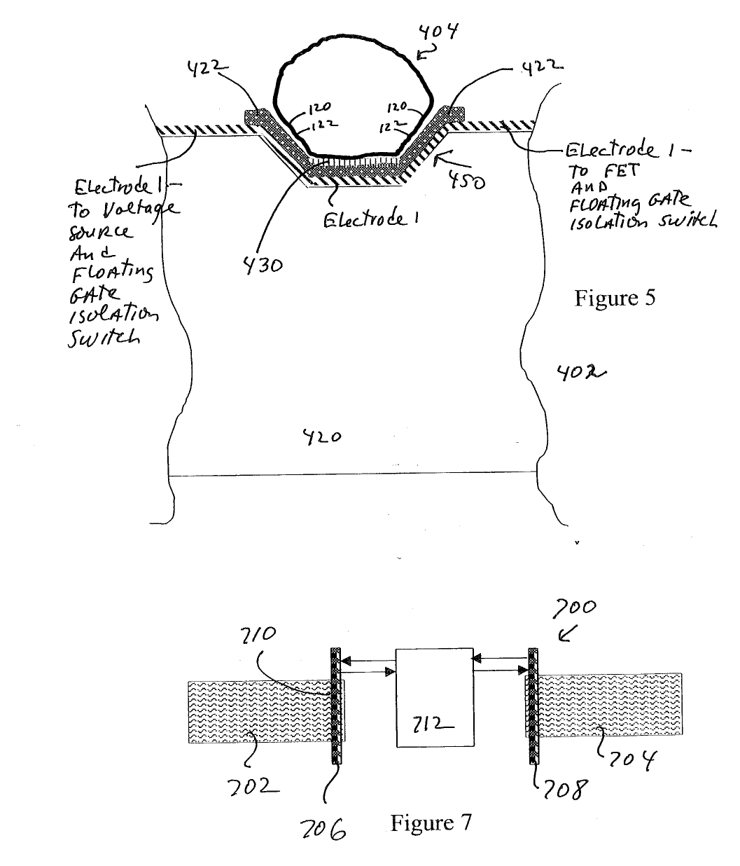

Illustrated in FIG. 5 in cross-section across the neuron 404 axis is an embodiment like that of system 400 of FIG. 4, which can both apply stimulation to the neuron, and can sense the electrochemical potential or “firing” of the neuron. In this regard, as shown in FIG. 5, the neuron 404 is guided to attach to an attachment layer 430 at the base of a channel 450 etched in the microcircuit substrate 420. The electrode 1, like the other electrodes 2, N-1, and N, may be connected through suitable control logic to a voltage source (not shown) to control the potential applied to its adjacent potassium-ion selective layer, and also may be connected to (or is itself) the floating gate of a field effect transistor sensing circuit, sometimes called a MEMFETWhen connected as a floating gate, the electrode 1 is disconnected from the power source, and is allowed to “float” freely. When connected to the voltage potential source, the electrode 1 is preferably disconnected from the FET floating gate function. The electrode 1 can be connected and disconnected by suitable solid state integrated circuit switching components, to either the floating gate, or the voltage/power source. When connected to be voltage/power source, the electrode can be used to stimulate the neuron, as previously described. When the electrode is isolated (to “float”) to serve as the floating gate of a FET transistor sensor, it is used to sense the electrochemical condition or “firing” status of the neuron. The field effect transistor (FET) is a conventional three terminal device in which the voltage on one terminal (the gate) controls the current between the other two (source and drain). An electric field over the channel (due to the potential at the gate) between the source and drain affects carriers at the surface of the channel, increasing or decreasing its conductivity. The conductivity can also be modulated by the drain-source voltage, resulting in several regions of I-V characteristics. As shown in FIG. 5, the ion selective layer 430, or preferably, zones 510 of the electrode system adjacent the ion-selective layer 430, may be a very thin, adherent layer of polylysine or other material which facilitates neuron binding and growth, which may partially cover the potassium-ion-selective layer 422 and electrode 1 as shown in FIG. 5, or which may be in zones adjacent the ion-selective layers. In use, the electrode 1 may be used to stimulate the neuron 404 in a manner similar to that described with respect to FIGS. 3 and 4. In this regard, the electrode may be quickly (e.g., 0.001 to 0.1 millisecond) pulsed to a positive potential value, which expels selected positive ions (such as K⁺) from the ion-selective reservoir zone 430, and locally decreases the internal potential of the adjacent neuron.

The floating gate electrochemical electrode 1 can also detect changes in potassium ion concentration, as well as membrane potential and electric field changes in the vicinity of the adjacent neuron, by disconnecting the electrode from its voltage/current source, and allowing it to float freely as the gate of the FET detector, to form an ion selective electrode sensor. Ion-selective-electrode (ISE) sensors are commonly used to measure the activity or concentration of various ions and metabolites present in biological fluids. Conventional ISE sensors employ potentiometric or amperiometric electrochemical processes which generate potential or current signals measuring the activity of a specific ion in a sample. For example, ISE sensors are typically used to determine chloride, potassium, lithium, calcium, magnesium, carbonate, hydrogen, and sodium ion content in biological fluids. Typically, the signal generated within the sensor is approximately linearly dependent on the logarithm of the activity of the ion of interest for potentiometric analyses. [see also, U.S. Pat. No. 4,502,938; H. Freiser, “Ion Selective Electrodes And Analytical Chemistry”, Vol. 2, Plenum Press, New York (1979); Anal. Sci. 4, 547 (1988); Anal. Chim. Acta 255, 35 (1991); J. Chem. Soc. Far. I82 1179 (1986); Clinical Chemistry 32, 1448 (1986); and SPIE 1510, 118 (1991); D. G. Davies et al., Analyst 1988, 113, 497-500; W. E. Morf, Studies in Analytical Chemistry, Punger, E. et al. (Eds.), Elsevier, Amsterdam (1981) p. 264; D. Ammann, Ion-Selective Microelectrodes, Springer (1986); U. Oesch, et al., Clin. Chem. 1986, 32. 1448; P. Oggenfuss, et al., Analytica Chim. Acta 1986, 180, 299; J. D. Thomas, R. J. Chem. Soc. Faraday Trans. I1986, 82, 1135].

If a plasticizer is used, dioctyl phthalate, dioctyl adipate, dioctyl sebacate, may be used to reduce the crystallinity of the organopolymeric matrix. Preferably, the plasticizer is also a copolymerizable material, so it becomes permanently integrated in the ion-selective storage layer in a non-leachable manner.

The ionophore may be present in the organic or inorganic matrix in any suitable form. A gel containing about 1-50% by weight percent valinomycin, about 20-30 weight percent polyvinyl chloride, and about 0-80% weight percent dibutyl sebacate. The ionophore gel is conveniently applied when dissolved in tetrahydrofuran solvent. The solvent may be removed by simple drying. Dibutyl sebacate acts as a plasticizer for the ionophore gel, allowing it to be built up in the form of a thin flexible membrane. For long-term stability and implantation, however, it is desirable to use internally plasticized polymers, and copolymerizable ionophores, in which a vinyl polymerizable group, such as an acryloyl or methacryloyl group is covalently attached to the valinomycin or other ionophore structure. The ion selective layer may also be cross-linked, and should best be covalently bonded or coupled to its substrate by a suitable silane or other suitable coupling agent for attachment adherence and long-term stability.

As discussed, the electrochemical electrodes may use a thin solid ion-selective layer or aqueous layer as storage reservoir for the selected ions. The ion selective layer may be used to sense ionic concentration in the standard manner, which may utilize a reference electrode (not shown). The electrode sensor may also sense the “firing” of the cell, in view of its proximity to the cell membrane. In operation, one surface of the sensing membrane is immersed in a biological sample solution of ions for which it is selective so that a potential develops across the membrane surface at the interface of the solution, and may be measured as a voltage by the underlying MOSFET system of the circuit 600. By comparing the voltage generated at the sensing membrane surface with that generated by a reference electrode, it is possible to calculate the concentration of the ionic species being sought. The desired selectivity may be accomplished by incorporating into the ion-selective agent such as an ionophore to increase the permeability of the layer to the specific ion. Generally, ion-selective layers may be conventionally formed from amorphous (e.g., plasticized) polymer matrix, such as polyvinyl chloride, which contains the ionophore selective for the ion of interest. The ionophore valinomycin produces a layer selective for potassium ions; trifluoroacetyl-p-butylbenzene or other trifluoroacetophenone derivatives are ionophores which produce ion-selective storage membranes for other ions.

As previously discussed, connexins may also be incorporated in or associated with the ionic reservoir in order to more directly provide selective ionic transport to stimulate or affect neuron or neuron target cell potential by ion transport through corresponding connexin pores on an adjacent neuron.

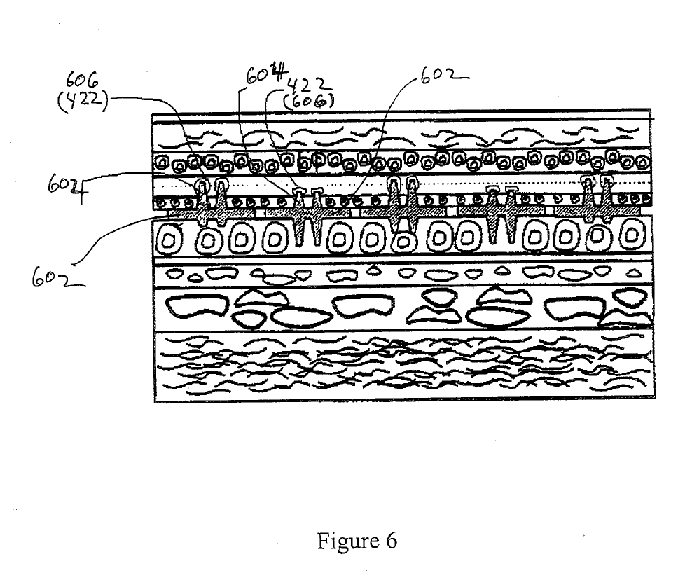

FIG. 6 is a cross-section of a retina showing photodiode retinal stimulation arrays 602 implanted in the subretinal space, with their stimulatory electrodes 604 penetrating into the sublamina B, and sublamina A locations of the inner plexiform of the eye, in a manner similar to the electrode arrays of U.S. Pat. No. 5,895,415, except that the metallic electrode tips are covered with potassium-ion-selective layers 606 like those 422 of FIGS. 4 and 5, and are operated primarily in a capacitive manner. In addition, the electrodes 604 are operated to produce low-voltage positive electrical pulses in response to incident light, e.g., in the manner of U.S. Pat. Nos. 5,411,540 and 5,944,747 The increased stimulatory efficiency of the capacitively driven potassium-selective electrodes, may permit very low power stimulation of retinal cells.

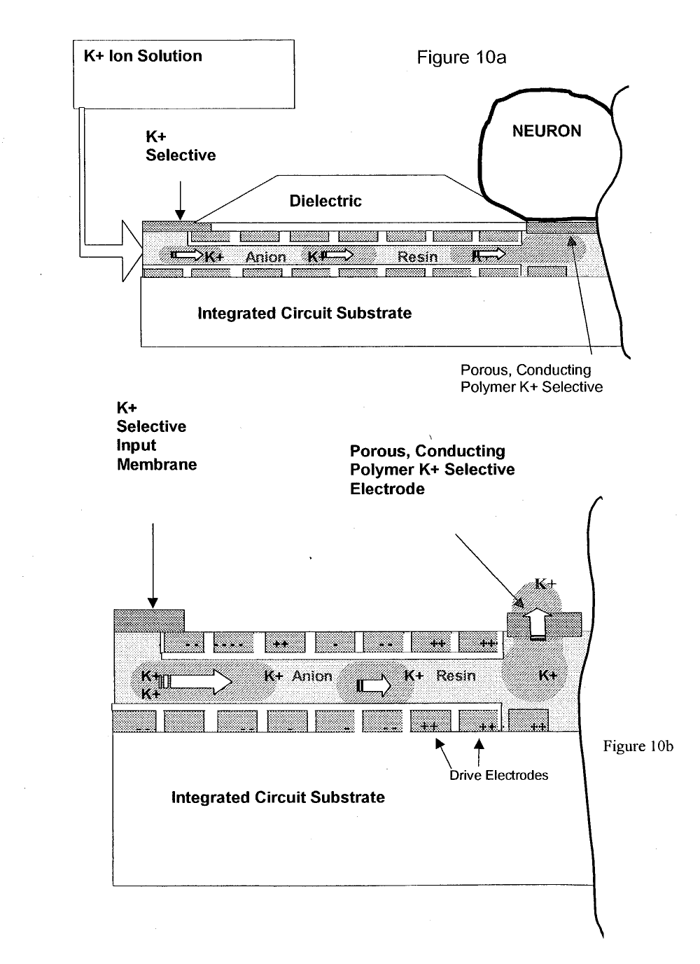

The “supply” of a selected ion may also be separated from the delivery of the ion to the cell membrane, so that operation does not depend on the re-equilibration of the ionophoric layer. As shown in FIG. 10a, a “peristaltic ion pump” may be used to manipulate, control and deliver selected ions to the cells and/or electrodes, more independently of the electrode environment:

The “peristaltic ion pump” functions to move the selected cations or anions by controlled application of transport electrode potentials in a manner similar to the operation of an integrated circuit “bucket brigade” or charge coupled device (CCD). The K+ ions may enter an anion resin channel (e.g. a 0.5 micron thick layer of Nafion 1100 product of Dupont) through a K+ selective input membrane, or may come from a separate reservoir (which may include a drug or neurotransmitter). Other types of micro/nano pumps may be used to deliver precise quantities of protective or restorative drugs. Many different drugs can be stored in small microarray cells adjacent specific sensor/stimulation electrodes, so that they can be applied as part of a test regimen, particularly to neurons which may be at the first stage of toxic stress.

By peristaltically applying + and − control voltages to the control electrodes in a moving three-phase manner, packets of K+ ions may be moved along the anion resin channel, to a porous neuron electrode, which can be “pulsed” to deliver the K+ ions, and any electrophoretically transported drug or neurotransmitter. The next “electrode phase” from FIG. 10a is shown in the following FIG. 10b:

The ion pump may be fabricated and operated in a manner similar to a surface channel CCD integrated electronic circuit, as shown, or may be provided for operation in a manner similar to a buried channel CCD. For a buried channel ion pump, an aqueous cationic resin polymer channel will have a negative “drain”, and a positive “drain” for an anionic resin transport zone. The potential within the channel is similarly then controlled by the drive electrodes to progressively transport selected cations or anions along the channel.

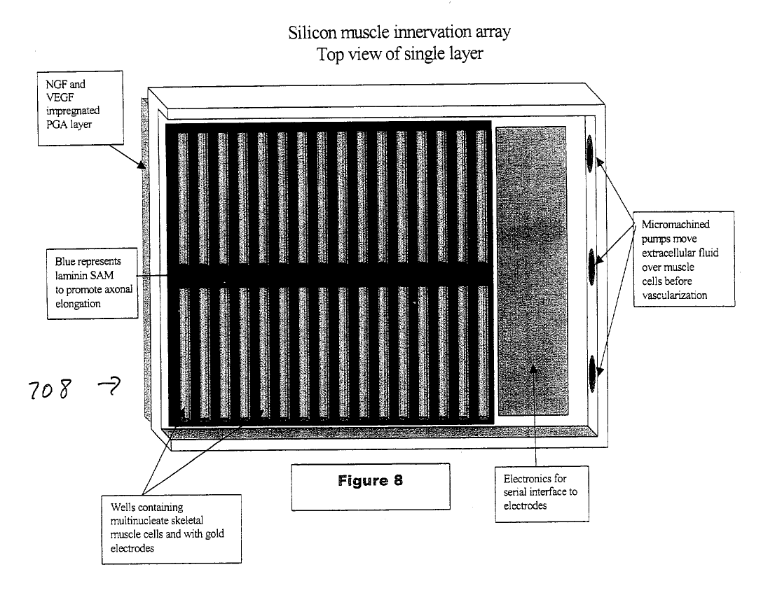

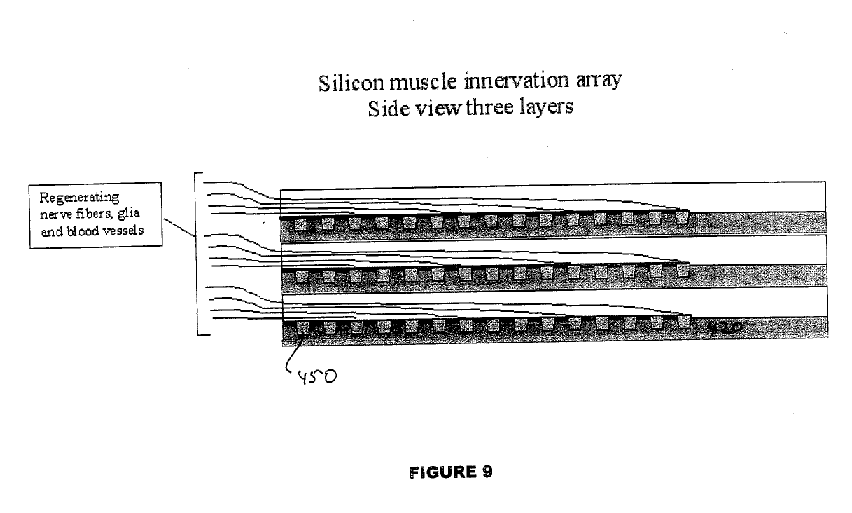

An example of a motor-control array 708 is shown in FIG. 8. This device guides bundles of efferent axons through planar tunnels to multinucleate muscle cells trapped in wells like those 450 of FIG. 5, where communication is established through both biological synapses and electrochemical stimulation/sensing systems with other cells and with the electrochemical electrodes of the system 708. One to one action potentials communicated to immobilized muscle cells are picked up by the electrodes and communicated to circuitry for amplification and electronic communication. Stacks of two dimensional patterns of wells containing muscle cells form a 3-D structure which axons innervate. Spacing is sufficient (>32 um) to allow axons to regenerate into the gaps. Electrodes with potassium-ion-selective layer coatings are exposed to neurons and/or target cells at the bottom of the wells, where they can sense action potentials in pre- or post-synaptic membranes, for processing, sensing and stimulation of cells. Other areas of the top surface shown in FIG. 8, including electronics are covered by a layer of polyimide followed by a layer of silicon. Three layers of the electrode system 708 are schematically shown in cross-section in FIG. 9. A SAM of glutaraldehyde-linked laminin is placed on top of the ion-selective membrane layer and/or around the wells to promote axonal adhesion and growth into the structure, as discussed with respect to FIG. 5. In order to prevent constriction of regenerated axons, a layer of PGA is deposited on the bottom of each chip. As regenerated axons and surrounding cells increase in size, the PGA will dissolve away, making more room. This layer is also impregnated with neural growth factor and vascular endothelial growth factor to encourage growth of neurons and blood vessels into the structure. In order to obtain nutrients for the muscle cells before the array becomes vascularized, micromachined pumps force extra-cellular fluid through the implant.

While the present invention has been described with respect to several specific embodiments, it will be appreciated that a wide variety of adaptations, variations, improvements, and implementations will be apparent or derived from the present disclosure, and are intended to be within the spirit and scope of the present invention as defined by the following claims. For example, selective reservoir layers of ionic materials such as dopamine, GABA, ACh, glycine, and other cell-stimulating ions and neurotransmitters may be provided for electronic modulation of the concentration of these materials adjacent to a neuron or neuron target cell membrane. Various ion-selective layer designs and combinations, bioengineering cell interface technologies, more complex electronic logic and control systems, electrochemical electrode patterns, designs arrays, and structures may be utilized, and a wide variety of current and future electronic and nanotechnology fabrication procedures and materials may be applied in the practice of this broad invention within its general scope.

What is claimed is:

1. A method for stimulating a neuron or neuron target cell which has a cell electropotential which is sensitive to the concentration of specific ion species at the exterior surface of its cell membrane or which has protein gate structures in its cell membrane which are sensitive or selective to one or more specific ion species, comprising of the steps of
providing at least one stimulating electrode and an ion-selective zone adjacent the cell membrane of the cell to be stimulated which selectively absorbs one or more of said ion species,
selectively absorbing said ion species in the ion-selective zone to create a reservoir of such ionic species in the ion-selective zone or beneath it in an aqueous layer, and
applying an electrical potential to the ion-selective zone to change the concentration of the ion species adjacent the cell membrane to induce a biological response in the cell.

2. A method accordance with claim 1 wherein said ion species is potassium, sodium, calcium, and/or chlorine ions, wherein the stimulating electrode is a metal, electrically conducting organopolymer, or semiconductor electrode, wherein the ionically selective zone is a layer of an organic or inorganic matrix containing an ionophore selective to the ion species, and wherein the potential applied to the ion-selective zone is between + or − three volts with respect to the cell exterior.

3. A method in accordance with claim 1 wherein the stimulating electrode is an electrically conducting organic polymer such as polypyrrole, polyaniline or polythiophene, and wherein the stimulating electrode itself also forms the ion-selective zone.

4. A method in accordance with claim 1 wherein the ion-selective zone is a potassium-ion selective layer having a thickness of from about 1 to about 20 microns, wherein potassium is selectively absorbed into the layer at a preferential ratio of at least about 5 times the amount of sodium ions absorbed into the layer, and wherein the potassium ion concentration adjacent the cell membrane is increased by at least 5 mM adjacent the cell membrane by the application of the electrical potential.

5. A method like that of claim 1, in which a cationic ion exchange resin layer (which is selective to cations over anions, but not primarily selective among cations) is used to form a cation-selective zone.

6. A method in accordance with claim 1 in which opposite polarities are applied to adjacent electrodes to produce electric field lines parallel to the cell membrane between such electrodes, and in which the pulsed application of stimulating potential to the cell membrane is peristaltically applied along the cell membrane by varying the potential along a series of electrodes disposed along the cell.

7. An ion-selective electrochemical synapse system for efficiently stimulating a functional neuron target cell or neuron target cell, comprising,
an electrically conductive electrode,
a solid ion-selective reservoir layer positioned between the electrode and the adjacent cell, which is highly selective to a specific ionic species to provide a voltage-controllable reservoir of the specific ions in the ion-selective-reservoir layer, and
a voltage source for varying the potential applied to the ion-selective reservoir layer to modulate (selectively expel from, or incorporate into) the amount of the selected ion in the selective layer and in a zone adjacent both the cell membrane and the ion-selective layer, to stimulate the target cell by a combination of electric field and ion concentration.

8. An electrochemical synapse system in accordance with claim 7 wherein the voltage source provides a pulsed potential in the range of from 0.5 to 3.0 volts with a rise time of less than 1 millisecond and for a duration of at least 1 millisecond, wherein the ions elective reservoir layer has a thickness of from about 2 to about 10 microns and an interconnected porosity of at least about 5 volume percent, wherein the ion-selective reservoir layer is positioned within about 10 microns of the cell membrane surface zone to be stimulated, and wherein the ion-selective layer is selective to potassium ions.
